(19) United States
(12) Patent Application Publication
(10) Pub. No.: US 2017/0200440 A1
(43) Pub. Date: Jul. 13, 2017

(54) ACOUSTIC ATTENUATION DEVICE AND METHODS OF PRODUCING THEREOF

(71) Applicant: Shu Chun Chen, Lexington, MA (US)

(72) Inventor: Shu Chun Chen, Lexington, MA (US)

(21) Appl. No.: 15/401,032

(22) Filed: Jan. 8, 2017

Related U.S. Application Data
(60) Provisional application No. 62/276,805, filed on Jan. 8, 2016.

(51) Int. Cl. G10K 11/162; A61F 11/08; A61F 11/14; G10K 13/00

(52) U.S. Cl. CPC G10K 11/162; G10K 13/00; A61F 11/08; A61F 11/14; A61F 2011/085

(57) ABSTRACT

Micro-fabricated acoustic attenuation devices are described. One such device includes 1) a substrate, 2) a movable diaphragm supported by springs that anchors to the substrate, and 3) a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound.

Another device includes 1) a substrate, 2) a movable diaphragm wherein the diaphragm has at least one hole on it, and 3) a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound. Methods of producing the micro-fabricated acoustic attenuation device are also described.

### CROSS-REFERENCE TO RELATED APPLICATIONS

This application claims priority to Provisional Application No: 62/276,805, filed on Jan. 8, 2016

### STATEMENT REGARDING FEDERALLY SPONSORED RESEARCH OR DEVELOPMENT

N/A

### BACKGROUND

Field of the Technology

The present invention relates to a passive micro-fabricated acoustic attenuation device and in particular may be used in conjunction with macro-sized acoustic devices such as ear plugs, ear phones, headphones, helmets, and microphone housings.

Background

Noise Induced Hearing Loss (NIHL) is one of the major avoidable occupational hazards, particularly in developing countries, where occupational and environmental noise remains the major risk factor for hearing impairment. Even in developed countries hearing impairment continues to remain a common health disorder, leaving a largely untapped market to be exploited. More than 120 million workers across the globe are exposed to dangerously high noise levels (over 85 dB). The Occupational Safety and Health Administration estimates that around 30 million people in the U.S. are exposed to dangerously loud noise levels in their day-to-day life, with those in metalworking, manufacturing, coalmines, dockyard (fishermen) and construction, and hospitality industries comprising the most highly risk-prone groups.

There is also a pressing need to develop a passive acoustic attenuation device that helps military personnel reducing the risk of developing tinnitus and noise-induced hearing loss by protecting against transient harmful impact noise from explosions or firearms while allowing for hearing mission critical communication with minimum attenuation and distortion. Tinnitus, often referred to as “ringing in the ears,” and noise-induced hearing loss can be caused by a one-time exposure to hazardous impulse noise, or by repeated exposure to excessive noise over an extended period of time. Using the proper ear protection can prevent irreparable damage to the eardrums.

Conventional ear plugs and over-the-ear muffs attenuate both harmful impact noise as well as the sound of normal speech. To date, non-linear membrane technology is by far, the most innovative passive approach to hearing protection. Such technology aims at providing non-linear noise attenuation (U.S. Pat. No. 8,249,285B2) such that the attenuation is higher for high level sounds than for lower level sounds. Such non-linear noise attenuating device comprises housing with a hollow passageway for passing external sound through a flexible membrane. Typically the flexible membrane is made of polyethylene or Teflon foil. The device has three regimes of operation: normal sound, threshold sound, and maximum sound. Under normal sound environment, sound pressure causes the flexible membrane to expand allowing user to hear ambient sound. On the other hand, when the sound level reaches a threshold value (125 dB), the flexible membrane hits a perforated over-stop restricting the membrane to expand. When the sound level exceeds the peak value (125-171 dB), the membrane expands further through the perforation thus attenuating non-linearly.

There are several shortcomings relating to the existing non-linear noise attenuation device. Most important of all, the membrane is not flexible enough to function at a low sound threshold value. Second, during the normal sound regime, the existing membrane attenuates greatly due to the thick membrane and distorts the signal tremendously due to the uneven membrane stress. Such attenuation distorts the signal making users difficult to hear and understand speech properly. Third, in the maximum sound regime, the existing membrane still deflects due to high membrane elasticity and thus attenuates ineffectively. Finally, since there is no quality control on membrane manufacturing (such as internal stress, and thickness), attenuation varies from device to device.

Thus, there exists a need to new approach for acoustic attenuation device that operates at a low sound threshold level providing a low, uniform attenuation at all frequencies below a threshold value, yet providing a higher and increasing level of attenuation for sound level above that threshold.

### BRIEF SUMMARY

The below summary is merely representative and non-limiting. The above problems are overcome, and other advantages may be realized, by the use of the embodiments.

This invention discloses a micro-fabricated passive acoustic attenuation device that will allow significant enhancement in the ability to optimize the detection of low level ambient sound without distortion while shunting off high level impact noise. Such acoustic attenuation device offers unique acoustic engineering capabilities allowing users to hear mission critical communication, while helping reduce the risk of developing tinnitus and noise-induced hearing loss. The significant of this invention is that it is a low-cost passive acoustic attenuation device that protects users against transient impact noise while allowing for ambient sound without minimum attenuation and distortion. The micro-fabricated acoustic attenuation device offers non-distorted acoustic performance on normal sound, but rejects harmful sound when the diaphragm of the device is restricted by an over-stop for further movement. It is believed that this acoustic attenuation device would start attenuating at least 30 dB of impact noise at lower sound threshold level such as 65 dB, and 85 dB, and also operates at 125 dB, 140 dB, 160 dB and 171 dB; and a Noise Reduction Rate (NRR) of 12 or less between 30 to 60 dB.

Various embodiments provides an acoustic attenuating device comprising an ear mold comprising a non-hollow passageway, and a micro-fabricated acoustic attenuation device interposed across the hollow or non-hollow passageway, wherein said micro-fabricated acoustic attenuation device comprising a movable diaphragm, and a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound. The sound pressure threshold is approximately 140 dB. Further, the sound pressure threshold is approximately 125 dB. Even further the sound pressure threshold is approximately 85 dB. The proliferated backplane has at least one hole. The proliferated backplane and movable diaphragm are but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations. The thickness of the micro-fabricated diaphragm is less than 10 micrometers. The thickness of the micro-fabricated diaphragm is less than 2 micrometers. The diaphragm can be bossed such that the middle of the diaphragm is thicker than its side. The air gap is less than 10 micrometers. The air gap is less than 2 micrometers. Moreover, dimples could be placed on either the side of the diaphragm that faces the backplane or the side of the backplane that faces the diaphragm. Furthermore, the surface of the said diaphragm and the said proliferated backplane that pressed on each other could be coated with an anti-stiction layer. The anti-stiction layer could be a self-assembled monolayer. The anti-stiction layer could be but not limited to dichlorodimethylsilane (DDMS) or 1H,1H, 2H,2H-Perfluorodecyltrichlorosilane (FDTS) or Hexamethyldisiloxane (HMDS).

A method of attenuating incoming sound comprising the steps: a) providing an ear mold comprising a non-hollow passageway, and b) providing a micro-fabricated sound attenuation device interposed across the hollow or non-hollow passageway, wherein said micro-fabricated sound attenuation device comprising a movable diaphragm, and a stationary proliferated backplane which is separated by an air-gap, whereby sound pressure causes the movable diaphragm to vibrate and when the sound exceeds threshold, the movable diaphragm deflects and presses against the proliferated backplane restricting further movement thus attenuates incoming sound. The proliferated backplane has at least one hole. The proliferated backplane and the movable diaphragm is but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations. The membrane can be bossed. Moreover, dimples could be placed on either the side of the diaphragm that faces the backplane or the side of the backplane that faces the diaphragm. Further, the surface of the said diaphragm and the said proliferated backplane that pressed on each other could be coated with an anti-stiction layer. The anti-stiction layer could be a self-assembled monolayer. The anti-stiction layer could be but not limited to dichlorodimethylsilane (DDMS) or 1H,1H, 2H,2H-Perfluorodecyltrichlorosilane (FDTS) or Hexamethyldisiloxane (HMDS).

Another embodiment provides an acoustic attenuating device comprising an ear mold comprising a hollow or non-hollow passageway, and a micro-fabricated acoustic attenuation device interposed across the hollow or non-hollow passageway, wherein said micro-fabricated acoustic attenuation device comprising a movable diaphragm unlike the diaphragm described in U.S. Pat. No. 8,249,285B2, whereby the movable diaphragm has at least one hole on it, and a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound. The proliferated backplane has at least one hole. The proliferated backplane and movable diaphragm but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations. The diaphragm can be bossed. Moreover, dimples could be placed on either the side of the diaphragm that faces the backplane or the side of the backplane that faces the diaphragm. Furthermore, the surface of the said diaphragm and the said proliferated backplane that pressed on each other is coated with an anti-stiction layer. The anti-stiction layer could be a self-assembled monolayer. The anti-stiction layer could be but not limited to dichlorodimethylsilane (DDMS) or 1H,1H, 2H,2H-Perfluorodecyltrichlorosilane (FDTS) or Hexamethyldisiloxane (HMDS).

A method of attenuating incoming sound comprising the steps: a) providing an ear mold comprising a hollow or non-hollow passageway, and b) providing a micro-fabricated sound attenuation device interposed across the hollow or non-hollow passageway, wherein said micro-fabricated sound attenuation device comprising a movable diaphragm, wherein the movable diaphragm has at least one hole on it, and a stationary proliferated backplane which is separated by an air-gap, whereby sound pressure causes the movable diaphragm to vibrate and when the sound exceeds threshold, the movable diaphragm deflects and presses against the proliferated backplane restricting further movement thus attenuates incoming sound. The proliferated backplane has at least one hole. The proliferated backplane and movable diaphragm are but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations. The membrane can be bossed. Moreover, dimples could be placed on either the side of the diaphragm that faces the backplane or the side of the backplane that faces the diaphragm. Further, the surface of the said diaphragm and the said proliferated backplane that pressed on each other could be coated with an anti-stiction layer. The anti-stiction layer could be a self-assembled monolayer. The anti-stiction layer could be but not limited to dichlorodimethylsilane (DDMS) or 1H,1H, 2H,2H-Perfluorodecyltrichlorosilane (FDTS) or Hexamethyldisiloxane (HMDS).

Yet in another embodiment provides an acoustic attenuating device comprising an ear mold comprising a hollow or non-hollow passageway, and a micro-fabricated acoustic attenuation device interposed across the hollow or non-hollow passageway, wherein said micro-fabricated acoustic attenuation device comprising a movable diaphragm unlike the diaphragm described in U.S. Pat. No. 8,249,285B2, whereby the movable diaphragm is anchored by springs to the stationary backplane, and a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound. The proliferated backplane has at least one hole. The proliferated backplane and movable diaphragm are but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations. Moreover, dimples could be placed on either the side of the diaphragm that faces the backplane or the side of the backplane that faces the diaphragm. Further, the surface of the said diaphragm and the said proliferated backplane that pressed on each other could be coated with an anti-stiction layer. The anti-stiction layer could be a self-assembled monolayer. The anti-stiction layer could be but not limited to dichlorodimethylsilane (DDMS) or 1H,1H, 2H,2H-Perfluorodecyltrichlorosilane (FDTS) or Hexamethyldisiloxane (HMDS).

A method of attenuating incoming sound comprising the steps: a) providing an ear mold comprising a hollow or non-hollow passageway, and b) providing a micro-fabricated sound attenuation device interposed across the hollow or non-hollow passageway, wherein said micro-fabricated sound attenuation device comprising a movable diaphragm, wherein the movable diaphragm is anchored by springs to the stationary backplane, and a stationary proliferated backplane which is separated by an air-gap, whereby sound pressure causes the movable diaphragm to vibrate and when the sound exceeds threshold, the movable diaphragm deflects and presses against the proliferated backplane restricting further movement thus attenuates incoming sound. The proliferated backplane has at least one hole. The proliferated backplane and movable diaphragm are but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations. Moreover, dimples could be placed on either the side of the diaphragm that faces the backplane or the side of the backplane that faces the diaphragm. Further, the surface of the said diaphragm and the said proliferated backplane that pressed on each other could be coated with an anti-stiction layer. The anti-stiction layer could be a self-assembled monolayer. The anti-stiction layer could be but not limited to dichlorodimethylsilane (DDMS) or 1H,1H, 2H,2H-Perfluorodecyltrichlorosilane (FDTS) or Hexamethyldisiloxane (HMDS).

### BRIEF DESCRIPTION OF THE SEVERAL VIEWS OF THE DRAWINGS

Various embodiments are illustrated by way of example, and not by way of limitation, in the Figures of the accompanying drawings and in which like reference numerals refer to similar elements and in which:

FIG. 1 shows the schematics of an embodiment of a macro-sized acoustic attenuation device.

FIG. 2 shows the cross-section of an embodiment of a macro-sized acoustic attenuation device.

FIG. 3 shows various embodiments of macro-sized acoustic attenuation device.

FIG. 4 shows the top (a) and cross sectional (b) view of a micro-fabricated acoustic attenuation device.

FIG. 5 shows the operation of a micro-fabricated acoustic attenuation device.

FIG. 6 shows the top (a) and cross sectional (b) view of another embodiment of a micro-fabricated acoustic attenuation device.

FIG. 7 illustrate a detailed diagrammatic cross-sectional process flow of a micro-fabricated acoustic attenuation device.

### DETAILED DESCRIPTION

Various embodiments are described in detail with reference to a few examples thereof as illustrated in the accompanying drawing. In the following description, numerous specific details are set forth in order to provide a thorough understanding of this disclosure. It will be apparent, however, to one skilled in the art, that additional embodiments may be practiced without some or all of these specific details. Additionally, some details may be replaced with other well-known equivalents. In other instances, well-known process steps have not been described in detail in order to not unnecessarily obscure the present disclosure.

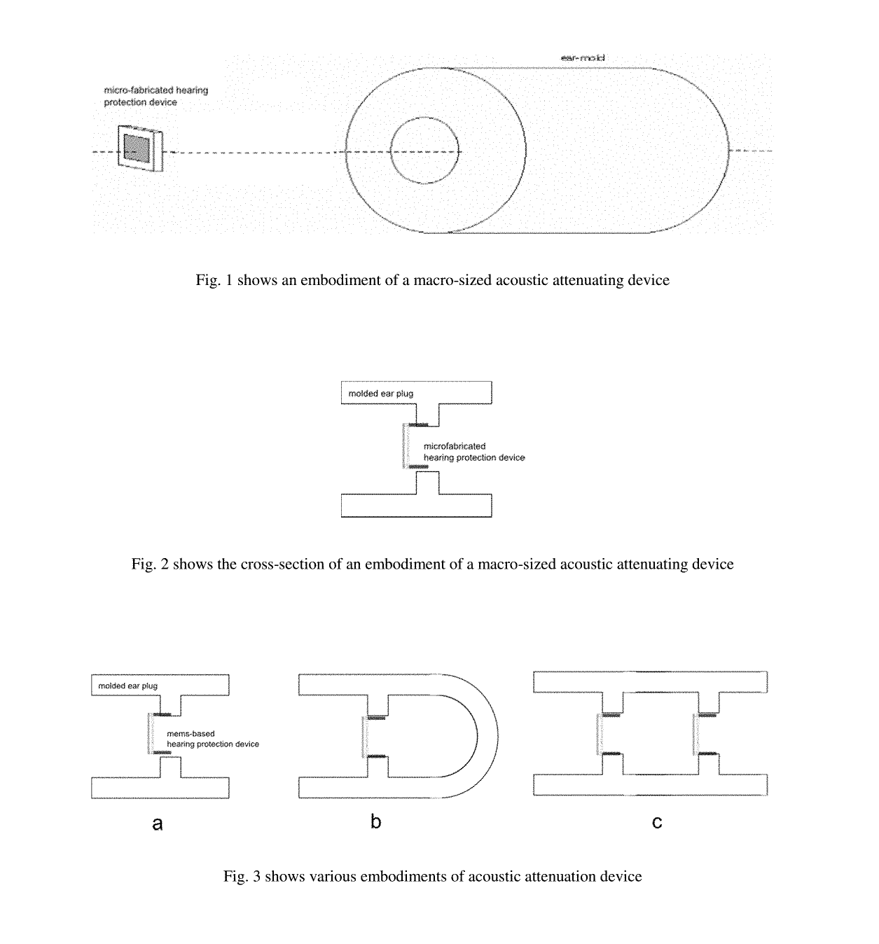

FIG. 1 shows the schematics of a macro-sized acoustic attenuation device featuring an ear-mold embedding a hollow passageway for passing external sound through a micro-fabricated acoustic attenuation device whereby the silicon chip is attached to the ear-mold. The assembly of such embodiment could be rather simple. The lightweight device is a passive non-linear attenuation device and does not contain any electronic components. FIG. 2 shows the cross-section of such acoustic attenuation device. In another embodiment, the micro-fabricated acoustic attenuation device could be attached to a fixture which in turn attached to the ear-mold.

The macro-sized acoustic attenuating device includes, but not limited to, ear plug, ear phone, helmet, and microphone housings. Design of the macro-sized acoustic attenuating device is not limited by the size, shape or structure shown in FIG. 1 and FIG. 2. Embodiment of a macro-sized ear plug can be in form of cylindrical foam or ear plug having triple-flange eartip to keep the device in place. These ear-plugs would be low-cost high-attenuation plastic ear plugs that are easy to insert and are in compliance with Foreign Objects and Debris (FOD) requirements in proximity with military aircraft and flight lines. Such rubber ear plug should be robust and compatible with long term use. FIG. 3 shows various embodiments of the macro-sized ear plug. In FIG. 3b, the ear plug is designed such as the passageway is non-hollow. In FIG. 3c, multiple micro-fabricated acoustic attenuation devices can be placed along the passageway.

### Micro-Fabricated Acoustic Attenuation Device

A major component of the invention is the micro-fabricated acoustic attenuation device which offers non-distorted acoustic performance on normal sound, but rejects harmful sound when its over-stop restrict further movement of the diaphragm. It is believed that this acoustic attenuation device would start attenuating at least 30 dB of impact noise at 65 dB, 85 dB, an continue operating at 125 dB, 140 dB, 160 dB and 171 dB; and a Noise Reduction Rate (NRR) of 12 or less between 30 to 60 dB.

A major advantage of this acoustic attenuation device is that it is micro-fabricated. The micro-fabricated acoustic attenuation device is manufactured in a batch mode using Micro Electro Mechanical System (MEMS) technology similar to the integrated circuit fabrication process used in microelectronic industry. Batch processing of the micro-fabricated acoustic attenuation device not only allows tight quality control, it also drives the manufacturing cost low as the volume of production increases.

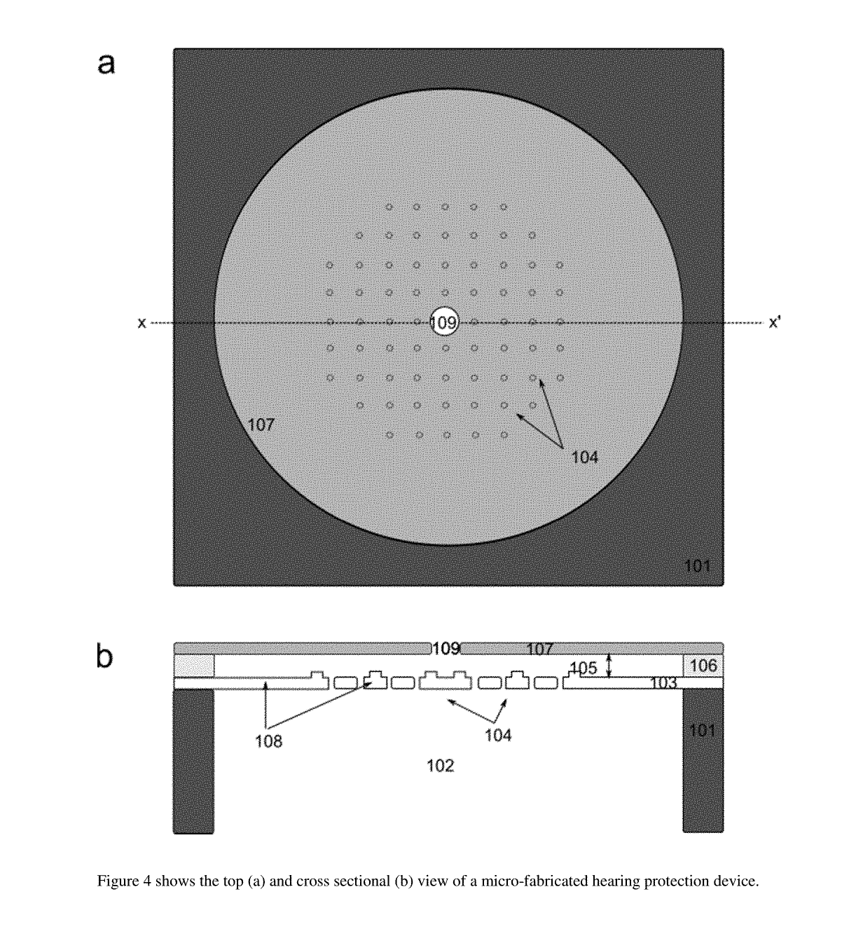

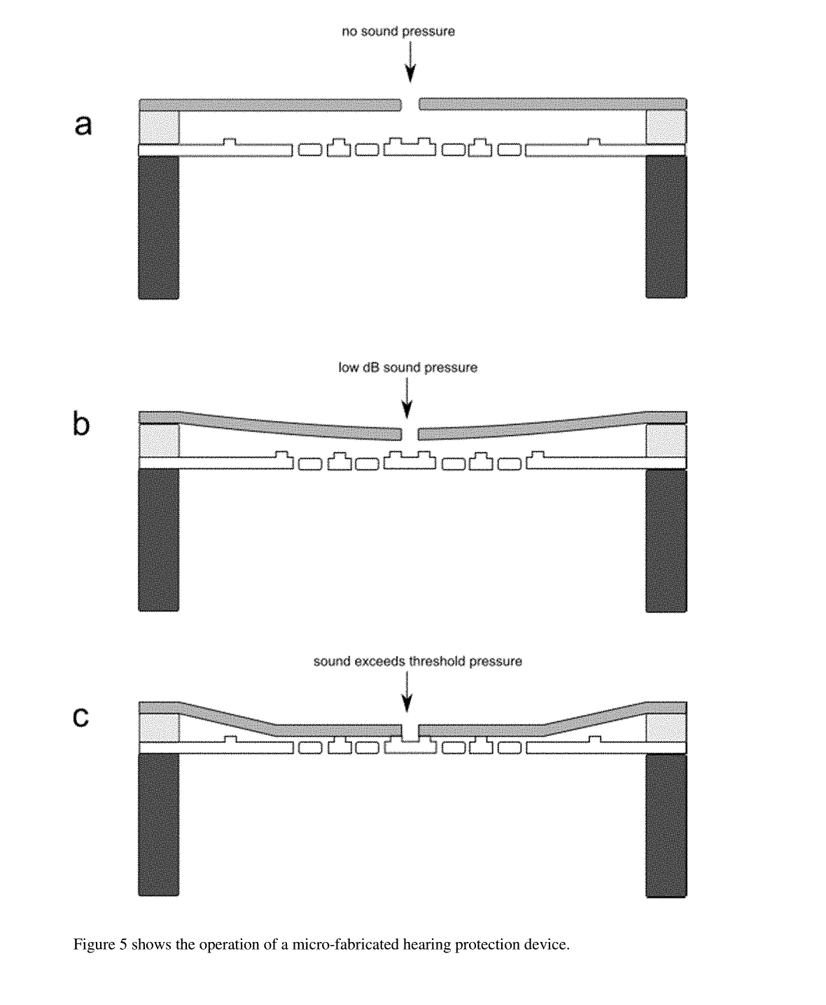

FIG. 4 shows the top (a) and cross sectional (b) view of a micro-fabricated acoustic attenuation device. In this embodiment, the device is constructed on top of silicon substrate with a rigid backplane. Next, a diaphragm is constructed as a suspended membrane on top of the rigid backplane separated by a micron-size air gap. The novelty of the micro-fabricated sound attenuation device is the suspended diaphragm can be patterned and etched to achieve certain specifications, unlike U.S. Pat. No. 8,249,285B2. The suspended diaphragm in FIG. 4 is patterned by micro-lithography and etched to form at least one hole on the diaphragm. Such pattern allows higher diaphragm elasticity and thus acoustic sensitivity such that the acoustic attenuation device can operate at a lower sound threshold level. Array of back-vent perforations are constructed on the backplane to prevent pressure buildup when the diaphragm is pushed toward the backplane.

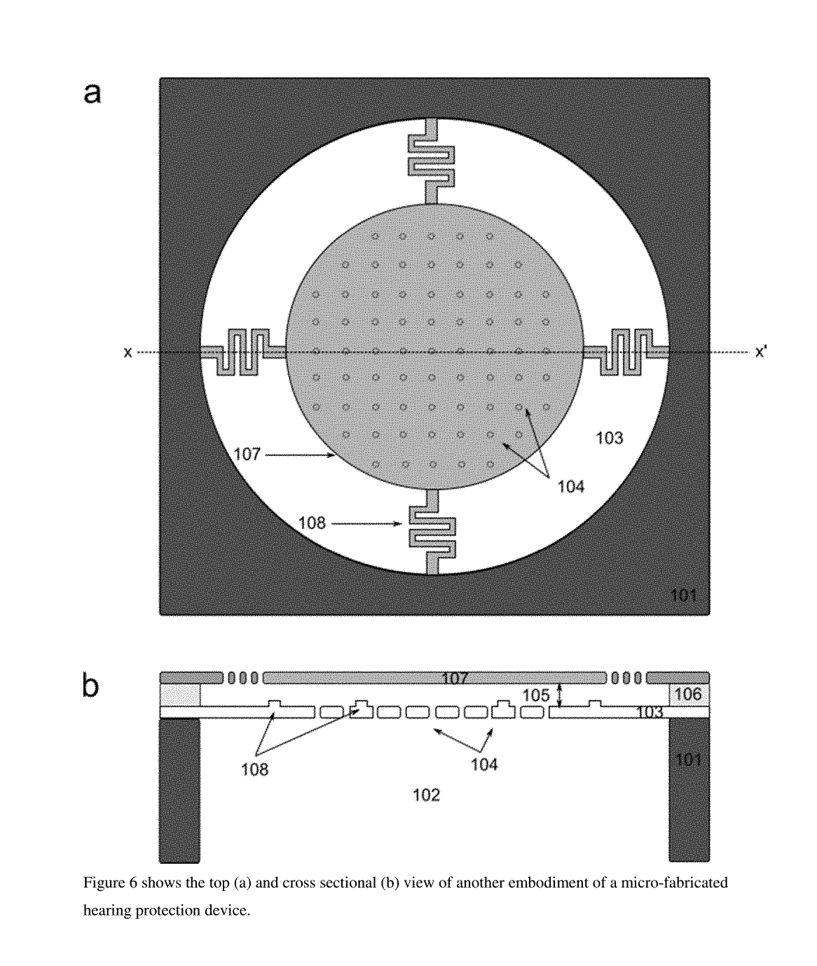

During the normal sound regime, incoming sound hits the sensing diaphragm. The sensing diaphragm (see FIG. 6b) vibrates with amplitude depending on the strength of the incoming sound. The membrane attenuates slightly due to the thin (several micrometer thick) membrane with little distortion due to the uniform and tensile stress of the diaphragm. Such minimum signal attenuation and distortion making users easy to hear and understand speech properly. In threshold sound regime (see FIG. 6c), the micro-fabricated diaphragm contacts the backplane prohibiting its further movement. Any incoming signal greater than threshold sound would completely land on the backplane thus restricting any sound vibration. The threshold sound is determined by the diaphragm material, diaphragm thickness, gap distance (distance between diaphragm and backplane). In maximum sound regime, the diaphragm would not deflect through the backplane vent hole due to high mechanical strength of the diaphragm and thick backplane and with proper design of small backplane vent hole size.

In order to achieve the thickness of the diaphragm and tight thickness tolerance, the diaphragm needs to be fabricated by thin film process. Selection of diaphragm material is also crucial since sensitivity increases tremendously with thin and low-tensile stress diaphragm. Under uniform tensile stress, the diaphragm would displace linearly with small perturbation of sound pressure. Thin film membrane materials such as doped polysilicon, un-doped polysilicon, p+ doped silicon, silicon nitride parylene, polyimide, negative photo-definable SU8 resin, metal, Teflon, polydimethylsiloxane (PDMS), poly(methyl methacrylate) (PMMA) or any combinations could be used. With high diaphragm sensitivity and minimal distortion, the micro-machined diaphragm shall maintain the ability of the user to detect, identify, and localize sound, with a goal of allowing for near-normal hearing in quiet environments.

FIG. 6 shows the top (a) and cross sectional (b) view of another embodiment of a micro-fabricated sound attenuation device. In this embodiment, the suspended diaphragm is supported by springs that anchored to the substrate with a rigid backplane. The springs design further increases sensitivity of the diaphragm to sound pressure. Springs are commonly used in field of MEMS sensor and actuator. Therefore the design of springs are commonly known to the art and are not described in detail here.

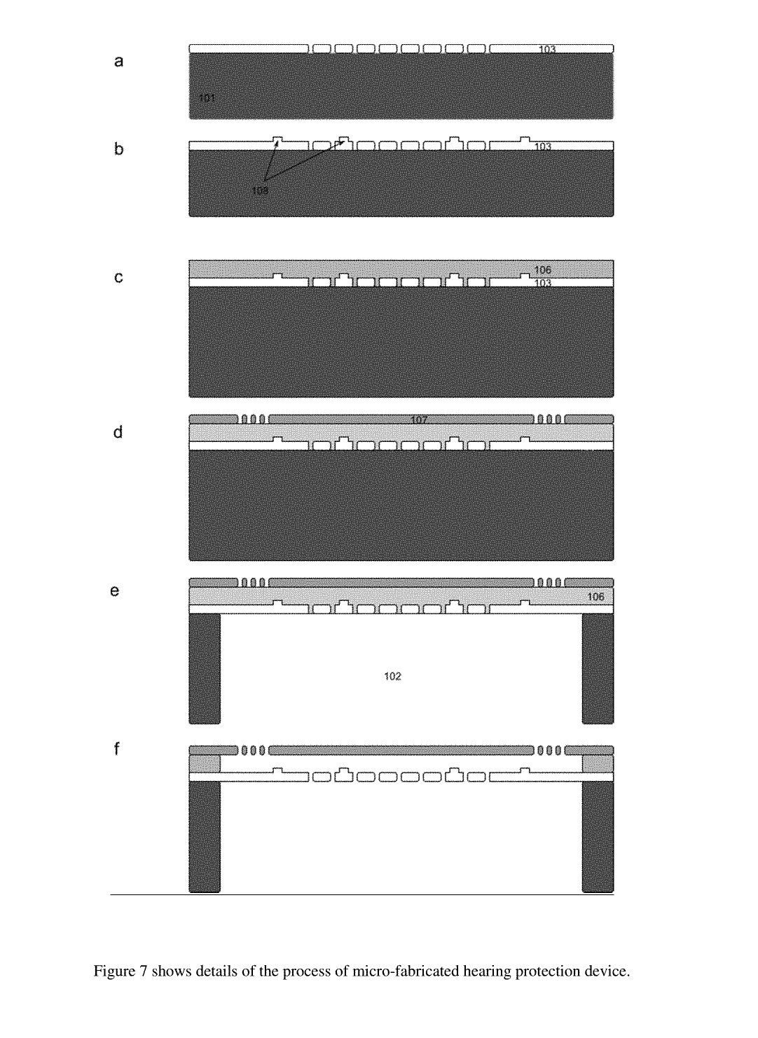

Details of the process of micro-fabricated acoustic attenuation device are shown in FIGS. 7a-7f. On a silicon oxide grown substrate (101), a silicon nitride or polysilicon film (103) is first deposited and patterned forming the backplane (FIG. 7a) and thickness of the backplane can be of several micrometers. The backplane could be selectively etched (FIG. 7b) to form small dimples (108). These dimples helps reducing stiction between the movable diaphragm and backplane. The use of dimples to reduce stiction is known to the art.

Shown in FIG. 7c, a several micrometer thick sacrificial layer (106) is next deposited defining the air-gap spacing. Sacrificial material could be silicon dioxide or polysilicon. Next a thin layer of thin film diaphragm material is formed. The diaphragm could be formed by low pressure chemical vapor deposition of low-stress polysilicon film (107) at elevated temperature (see FIG. 7d). The polysilicon film could be doped. The polysilicon could next be annealed at high temperature such as 1000 C to remove as much residual stress as possible. The polysilicon layer is then patterned and etched using reactive ion etching of Sulfur Hexaflouride (SF6) to form diaphragm layer. The diaphragm film could be a combination of silicon nitride, silicon oxide and polysilicon to form a stress balancing film. The diaphragm film could be deposited using room temperature deposition of plasma polymerization of parylene, followed by oxygen plasma etching forming spring-anchored diaphragm. SU-8 could be spin casted and photo-defined to be bossed structure at top of the diaphragm.

The backside of the wafer is then patterned and then etched in deep reactive ion etching (DRIE) until it stops on the backside of the backplane (see FIG. 7e). The substrate could be singulated in separated die at this point. When sacrificial material is silicon dioxide, the substrate could be immersed in hydrofluoric acid, such that the hydrofluoric acid removes the sacrificial oxide layer from the backside (see FIG. 7f). The sacrificial oxide could also be removed by vapor hydrofluoric acid etching. After sacrificial etching, the substrate could undergo supercritical point drying to prevent in-process stiction. When sacrificial material is polysilicon, the substrate can be exposed to Xenon difluoride (XeF2) etching. Since Xenon difluoride etching is done in gaseous phase, such drying etching scheme can prevent in-process stiction. To further prevent future in-use stiction, the substrate could then be coated with an anti-stiction layer. The anti-stiction layer could be a self-assembled monolayer. The anti-stiction layer could be dichlorodimethylsilane (DDMS) or 1H,1H, 2H,2H-Perfluorodecyltrichlorosilane (FDTS) or Hexamethyldisiloxane (HMDS). The substrate could be diced before or after the coating of the anti-stiction layer.

What is claimed is:

1. An acoustic attenuation device comprising
a. an ear mold comprising a hollow or non-hollow passageway, and
b. at least one micro-fabricated acoustic attenuation device interposed across the passageway,
wherein said micro-fabricated acoustic attenuation device comprising
1 a substrate,
2 a movable diaphragm supported by springs that anchor to the substrate, and
3 a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound.

2. The sound pressure threshold according to claim 1 is approximately 85 dB.

3. The movable diaphragm according to claim 1 is non-expandable into holes of the said proliferated backplane.

4. The thickness of the micro-fabricated diaphragm according to claim 1 is less than 10 micrometers.

5. The micro-fabricated diaphragm according to claim 1 is but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations.

6. The said diaphragm according to claim 1 could be bossed such that the middle of the membrane is thicker than the peripherals.

7. The air gap according to claim 1 is less than 10 micrometers.

8. Further to claim 1, the surface of the said diaphragm that faces the backplane or the surface of the said backplane that faces the diaphragm has dimples on it to reduce stiction.

9. Further to claim 1, the surface of the said diaphragm and the said proliferated backplane that pressed on each other is coated with an anti-stiction layer which could be but not limited to dichlorodimethylsilane (DDMS) or 1H,1H, 2H,2H-Perfluorodecyltrichlorosilane (FDTS) or Hexamethyldisiloxane (HMDS).

10. An acoustic attenuation device comprising
c. an ear mold comprising a hollow or non-hollow passageway, and
d. at least one micro-fabricated acoustic attenuation device interposed across the passageway,
wherein said micro-fabricated acoustic attenuation device comprising
1 a substrate,
2 a movable diaphragm wherein the said diaphragm has at least one hole on it, and
3 a stationary proliferated backplane which is separated by an air gap, whereby sound pressure causes the movable diaphragm to vibrate and when the sound exceeds threshold, the movable diaphragm deflects and presses against the proliferated backplane restricting further movement thus attenuates incoming sound.

11. The sound pressure threshold according to claim 10 is approximately 85 dB.

12. The movable diaphragm according to claim 10 is non-expandable.

13. The micro-fabricated diaphragm according to claim 10 is but not limited to un-doped polysilicon, doped polysilicon, silicon, doped silicon, silicon nitride, silicon oxide, metal, polymer, parylene, polyimide, negative photo-definable SU8 resin, metal, Teflon, polydimethylsiloxane (PDMS), poly(methyl methacrylate) (PMMA) or any combinations.

14. The air gap according to claim 10 is less than 10 micrometers.

15. Further to claim 10, the surface of the said diaphragm that faces the backplane or the surface of the said backplane that faces the diaphragm has dimples on it to reduce stiction.

16. Further to claim 10, the surface of the said diaphragm and the said proliferated backplane that pressed on each other is coated with an anti-stiction layer which could be but not limited to dichlorodimethylsilane (DDMS) or 1H,1H, 2H,2H-Perfluorodecyltrichlorosilane (FDTS) or Hexamethyldisiloxane (HMDS).

17. A method of making a micro-fabricated acoustic attenuation device comprising the steps:
Providing a substrate,
Providing a movable diaphragm supported by springs that anchor to the substrate, and
Providing a stationary proliferated backplane which is separated by an air-gap, whereby sound pressure causes the movable diaphragm to vibrate and when the sound exceeds threshold, the movable diaphragm deflects and presses against the proliferated backplane restricting further movement thus attenuates incoming sound.

18. Further to claim 17, the surface of the said diaphragm that faces the backplane or the surface of the said backplane that faces the diaphragm has dimples on it to reduce stiction.

19. Further to claim 17, the surface of the said diaphragm and the said proliferated backplane that pressed on each other is coated with an anti-stiction layer which could be but not limited to dichlorodimethylsilane (DDMS) or 1H,1H, 2H,2H-Perfluorodecyltrichlorosilane (FDTS) or Hexamethyldisiloxane (HMDS).

20. Further to claim 19, the anti-stiction layer could be applied after the said micro-fabricated acoustic attenuation device is singulated in die form.
